(19) United States
(12) Patent Application Publication
(10) Pub. No.: US 2022/0056556 A1
(43) Pub. Date: Feb. 24, 2022

(54) BRINE AND METHOD FOR PRODUCING SAME

(71) Applicant: LI-Technology Pty Ltd., Belmont (AU)

(72) Inventors: Gary Donald JOHNSON, Belmont (AU); Mark Daniel URBANI, Wattle Grove (AU); Nicholas John VINES, High Wycombe (AU)

(73) Assignee: LI-Technology Pty Ltd., Belmont (AU)

(21) Appl. No.: 17/275,941

(22) PCT Filed: Sep. 25, 2019
(86) PCT No.: PCT/AU2019/051024

§ 371 Date: Mar. 12, 2021

(30) Foreign Application Priority Data
Oct. 10, 2018 (AU) 2018903825

(51) Int. Cl. C22B 26/10; C22B 3/26; B01D 11/04; C07C 51/41

(52) U.S. Cl. CPC C22B 26/10; C22B 3/262; C07C 51/41; B01D 11/0492; B01D 11/0488

(57) ABSTRACT

A method to produce a brine from mixed alum salts, the method comprising the steps of: (i) Dissolving or pulping alum salts (1) containing rubidium alum, cesium alum and/or potassium alum in water or a recycled liquor and adding a neutralising agent to precipitate (20) aluminium as aluminium hydroxide and some sulfate; (ii) Passing the product of step (i) to a solid liquid separation stage (21) to remove precipitated solids (5) from step (i); (iii) A decant or filtrate (6) from step (ii) is passed to a solvent extraction stage (24-27) whereby any contained cesium and rubidium is selectively extracted into the organic phase to form a loaded organic solution (16); (iv) Contacting the loaded organic solution (16) of step (iii) with a scrub solution (17), which is at a pH lower than the extraction pH, to effectively scrub co-loaded potassium from the organic phase; (v) Contacting the scrubbed organic (19) of step (iv) with formic acid (20) to strip cesium and rubidium from the organic, the stripped cesium and rubidium forming a cesium and/or rubidium sulfate brine (21); and (vi) Recycling the stripped organic (22) of step (v) to the extraction stage (24-27).

### FIELD OF THE INVENTION

The present invention relates to a brine and a method for producing same. More particularly, the brine of the present invention is a cesium and rubidium formate brine.

Further and more particularly, the method of the present invention relates to the recovery of cesium and rubidium for the production of a brine, the cesium and rubidium being recovered from alum salts produced from the leaching of lithium containing mica.

The method of the present invention is intended, in one form, for use in the recovery of cesium and rubidium, and separation of such from other monovalent cations, such as lithium, potassium and sodium, and anions such as sulfate and chloride, by solvent extraction. The process of solvent extraction employed utilises phenolic functionalities, such as long chain phenols, to selectively extract cesium and rubidium from solution. Cesium and rubidium, present in the loaded organic, are recovered by stripping with formic acid to form cesium and rubidium formate brine.

### BACKGROUND ART

The Applicant's International Patent Application PCT/AU2015/000608 (WO 2016/054683) titled ‘Recovery Process’, the entire content of which is hereby incorporated by reference, describes a hydrometallurgical process for the extraction and recovery of lithium from lithium containing micas. In this process lithium and other metals contained in lithium micas, such as rubidium, cesium, potassium and aluminium, are extracted by leaching in sulphuric acid solution. Rubidium, cesium and potassium are separated from lithium by the selective crystallisation of rubidium, cesium and potassium alum salts, which are double salts of rubidium sulfate and aluminium sulphate, cesium sulfate and aluminium sulfate and potassium sulfate and aluminium sulfate.

The separation efficiency of rubidium, cesium and potassium from lithium is high since lithium sulfate does not form a double salt with aluminium sulfate. In the described process, the recovery of rubidium, cesium and potassium is by way of a series of precipitation and crystallisation processes to produce potassium sulfate product and a mixed rubidium and cesium product. Initially the mixed alum salts are re-dissolved and aluminium is precipitated as aluminium hydroxide by increasing the pH. The monovalent cations are subsequently separated and recovered by selective crystallisation of their respective sulfates.

Whilst the separation of potassium sulfate, rubidium sulfate and cesium sulfate is possible by selective crystallisation, separation in this manner does not have a high efficiency. For example, the potassium sulfate will contain some rubidium sulfate and the mixed rubidium and cesium sulfate will contain some potassium sulfate. The efficiency of this separation can be improved by conducting re-crystallisation stages, although this brings added cost.

The potassium sulfate, rubidium sulfate and cesium sulfate products of the prior art processes are not readily saleable due to their impurity. For example, impurities including chloride, sodium and the like are likely to be present despite the use of techniques such as crystallisation.

The present invention has as one object thereof to overcome substantially the abovementioned problems associated with the prior art, or to at least provide a useful alternative thereto.

The preceding discussion of the background art is intended to facilitate an understanding of the present invention only. It should be appreciated that the discussion is not an acknowledgement or admission that any of the material referred to formed part of common general knowledge as at the priority date of the application.

Throughout the specification, unless the context requires otherwise, the word “comprise” or variations such as “comprises” or “comprising”, will be understood to imply the inclusion of a stated integer or group of integers but not the exclusion of any other integer or group of integers.

Throughout the specification, unless the context requires otherwise, the term “relatively” or variations thereof, when used in respect of a level of purity, is to be understood to refer to its relation to something achievable under the processes or in the products of the prior art. With regard to the use of the specific terms “relatively pure cesium formate brine” and “relatively pure rubidium formate brine” this references a specific gravity (SG) of at least about 1.7. It is preferable also that chloride and sulfate levels in such brines be considered “low”, which is to be understood as being low relative to similar products achievable by the processes, and in the products, of the prior art.

### DISCLOSURE OF THE INVENTION

In accordance with the present invention there is provided a method to produce a brine from mixed alum salts, the method comprising the steps of:
(i) Dissolving or pulping alum salts containing rubidium alum, cesium alum and/or potassium alum in water or a recycled liquor and adding a neutralising agent to precipitate aluminium as aluminium hydroxide and some sulfate,
(ii) Passing the product of step (i) to a solid liquid separation stage to remove precipitated solids from step (i):
(iii) A decant or filtrate from step (ii) is passed to a solvent extraction stage whereby any contained cesium and rubidium is selectively extracted into the organic phase to form a loaded organic,
(iv) Contacting the loaded organic solution of step (iii) with a scrub solution, which is at a pH lower than the extraction pH, to effectively scrub co-loaded potassium from the organic phase,
(v) Contacting the scrubbed organic of step (iv) with formic acid to strip cesium and rubidium from the organic, the stripped cesium and rubidium forming a cesium and/or rubidium sulfate brine; and
(vi) Recycling the stripped organic of step (v) to the solvent extraction stage (iii).

Preferably, potassium or sodium hydroxide may be added to maintain pH in the solvent extraction stage (iii) and thereby increase the extraction efficiency of rubidium and cesium. Still preferably, the active component of the organic comprises a phenolic functionality. Still further preferably, the extraction order is Cs>Rb>K>Li>Na.

The active component of the organic is, in one form of the present invention, a para alkyl substituted phenol. Preferably, the alkyl substituent contains from 9-20 carbon atoms and includes nonylphenol and dodecylphenol.

Preferably, the raffinate produced from step (iii), which may contain soluble extractant due to the high pH of the extraction stage, is contacted with organic solution and acidified liquor to recover soluble extract to the organic phase.

Still preferably, the raffinate post acidification, which contains a relatively high potassium rubidium and potassium/cesium ratio, is passed to a crystalliser to recover potassium sulfate. The solid potassium sulfate is separated from the crystallisation slurry by a solid liquid separation stage, such as a filter. The filtrate can be recycled to step (i).

Preferably, the aqueous scrub solution from step (iv), which contains potassium and some rubidium and cesium, is recycled to the extraction stage step (iii) to recover cesium and rubidium.

In a further form of the present invention cesium is separated from rubidium and potassium in an additional, initial solvent extraction stage. In this process, cesium is extracted from the solution prepared in step (i) in an extraction stage by which the pH and/or organic aqueous flowrates are controlled to limit the co-extraction of rubidium and potassium.

Preferably, the loaded organic, which contains cesium and some rubidium, is passed to a separate scrubbing stage conducted at a pH lower than the extraction pH, to thereby scrub co-loaded rubidium from the organic solution. The scrub solution is recycled to the extraction stage. The scrubbed organic is stripped with formic acid to produce a relatively pure cesium formate brine.

The raffinate produced from the initial solvent extraction stage is subject to extraction in accordance with step (iii) and the subsequent scrubbing and stripping stages in accordance with steps (iv) and (v) to produce a relatively pure rubidium formate brine.

In accordance with the present invention there is further provided a brine containing one or both of cesium formate and rubidium formate produced by the method described above.

Preferably, the brine has a specific gravity of greater than about 1.7.

In one form of the present invention the brine containing one or both of cesium formate and rubidium formate is used as a completion or drilling fluid.

### BRIEF DESCRIPTION OF THE DRAWINGS

The brine and method of producing that brine of the present invention will now be described, by way of example only, with reference to two embodiments thereof and the accompanying drawings, in which:—

FIG. 1 is a flow sheet depicting a hydrometallurgical process for the recovery of a mixed cesium and rubidium formate brine by solvent extraction and recovery of potassium sulfate by crystallisation in accordance with the present invention;

FIG. 2 is a variation of the flow sheet of FIG. 1 depicting a hydrometallurgical process for the recovery of separate cesium formate and rubidium formate brines by solvent extraction and recovery of potassium sulfate by crystallisation;

FIG. 3 is a graph showing metal extraction vs pH for a solvent extraction step using 40% nonylphenol in Shellsol 2046™. The data indicates excellent selectivity for cesium over rubidium and potassium and relatively good selectivity for rubidium over potassium;

FIG. 4 is a graph showing metal extraction vs pH for a solvent extraction step 10% nonylphenol in Shellsol 2046™, wherein the data indicates excellent selectivity for cesium over rubidium and potassium; and

FIG. 5 is a McAbe Thiele diagram showing the cesium, rubidium and potassium content of an aqueous solution and the cesium, rubidium and potassium content of a strip liquor used to strip an organic solution.

### BEST MODE(S) FOR CARRYING OUT THE INVENTION

In the hydrometallurgical processing of cesium, rubidium and potassium containing alum salts of the prior art, the cations are separated by initially precipitating aluminium from solution followed by selective crystallisation of the monovalent sulfate salts. In such a single stage crystallisation process, potassium sulfate is contaminated with the sulfates of the monovalent salts. A re-crystallisation process is required to improve the purity of the potassium sulfate product.

Rubidium and cesium may be precipitated as a mixed sulfate salt, however a further process is required to convert these salts to formate brines. This can be achieved by reacting the salts with calcium formate to produce gypsum and formate brine. This process may reduce the recovery of the cesium and rubidium to the brine and may further result in contamination.

The process of the present invention utilises solvent extraction to selectivity extract and separate cesium and rubidium from potassium. These metals can then be recovered by stripping loaded organic with formic acid, producing a formate brine.

The inventors believe the present invention to be advantageous over prior art methods as solvent extraction enables improved selectivity and separation of cesium, rubidium and potassium, and improved purity of the brines.

Solvent extraction of cesium and rubidium can be achieved with phenol extractants as follows:

2R—OH+Cs₂SO₄→2R—OCs+H₂SO₄

2KOH+H₂SO₄→K₂SO₄

Cesium and rubidium are extracted via an ion exchange mechanism with phenol in which the proton from the hydroxyl group of the phenol is exchanged for a metal cation. Advantageously, so as to increase the extraction extent of the metal cations, the free acid produced is neutralised. This can be achieved using any water soluble base, such as potassium hydroxide, sodium hydroxide, potassium carbonate, sodium carbonate and the like.

The stoichiometry of the equations shows one mole of cesium is loaded per mole of phenol, however a higher phenol:metal ratio is required, in which un-complexed phenol molecules solvate the phenol-cesium complex.

The present invention provides a method to produce a brine from mixed alum salts, the method comprising the steps of:
(i) Dissolving or pulping alum salts containing rubidium alum, cesium alum and/or potassium alum in water or a recycled liquor and adding a neutralising agent to precipitate aluminium as aluminium hydroxide and some sulfate,
(ii) Passing the product of step (i) to a solid liquid separation stage to remove precipitated solids from step (i):
(iii) A decant or filtrate from step (ii) is passed to a solvent extraction stage whereby any contained cesium and rubidium is selectively extracted into the organic phase to form a loaded organic,
(iv) Contacting the loaded organic solution of step (iii) with a scrub solution, which is at a pH lower than the extraction pH, to effectively scrub co-loaded potassium from the organic phase,
(v) Contacting the scrubbed organic of step (iv) with formic acid to strip cesium and rubidium from the organic, the stripped cesium and rubidium forming a cesium and/or rubidium sulfate brine; and
(vi) Recycling the stripped organic of step (v) to the extraction stage (iii).

Potassium hydroxide may be added to maintain pH in the solvent extraction stage (iii) and thereby increase the extraction efficiency of rubidium and cesium. The active component of the organic comprises a phenolic functionality and the extraction order is Cs>Rb>K>Li>Na.

The active component of the organic is, in one form of the present invention, a para alkyl substituted phenol. The alkyl substituent contains from 9-20 carbon atoms and includes nonylphenol and dodecylphenol.

The active component of the organic is combined with other organic molecules to act as synergists for metal extraction or third phase modification. These other organic molecules comprise phosphorus containing organic compounds, including but not limited to long chain phosphates, phosphoric acid, phosphonic acid and phosphinic acid.

The raffinate produced from step (iii), which may contain soluble extractant due to the high pH of the extraction stage, is contacted with organic solution and acidified liquor to recover soluble extract to the organic phase.

The raffinate post acidification, which contains a relatively high potassium rubidium and potassium/cesium ratio, is passed to a crystalliser to recover potassium sulfate. The solid potassium sulfate is separated from the crystallisation slurry by a solid liquid separation stage, such as a filter. The filtrate can be recycled to step (i).

The aqueous scrub solution from step (iv), which contains potassium and some rubidium and cesium, is recycled to the extraction stage step (iii) to recover cesium and rubidium.

In a further form of the present invention cesium is separated from rubidium and potassium in an additional, initial solvent extraction stage. In this process, cesium is extracted from the solution prepared in step (i) in an extraction stage by which the pH and/or organic aqueous flowrates are controlled to limit the co-extraction of rubidium and potassium.

The loaded organic, which contains cesium and some rubidium, is passed to a separate scrubbing stage conducted at a pH lower than the extraction pH, to thereby scrub co-loaded rubidium from the organic solution. The scrub solution is recycled to the extraction stage. The scrubbed organic is stripped with formic acid to produce a relatively pure cesium formate brine.

The raffinate produced from the initial solvent extraction stage is subject to extraction as per step (iii) and the subsequent scrubbing and stripping stages as per steps (iv) and (v) to produce a relatively pure rubidium formate brine.

A brine containing one or both of cesium formate and rubidium formate produced by the method described above is a further feature of the present invention. This brine has a specific gravity of at least about 1.7.

It is possible that some level of, say, potassium formate may be present in the brine of the present invention. However, it is preferable that there be a relatively high ratio of cesium and/or rubidium to the potassium present. The Applicants have determined however that the specific gravity of the brine of the present invention should be at least about 1.7 to ensure the appropriate relative levels of cesium and/or rubidium to the potassium present, preferably with low chloride and sulfate levels. In this manner the brine can comprise a mixture of cesium, rubidium and potassium formate whilst maintaining a specific gravity of greater than about 1.7, as cesium formate has a relatively high SG (up to 2.2) and potassium formate has a relatively low SG (at about 1.6), whilst rubidium falls therebetween.

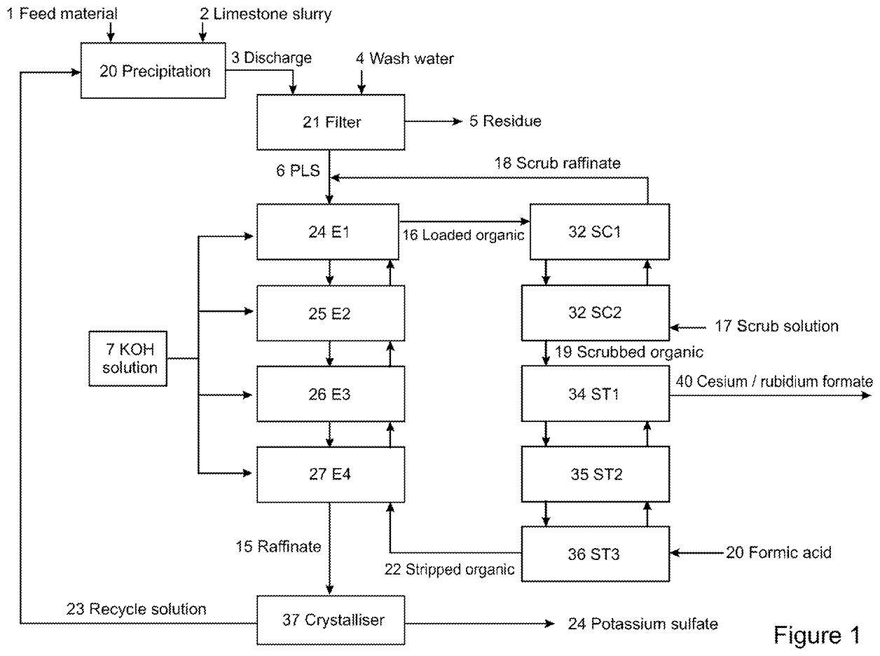

In FIG. 1 there is shown a method to produce a brine in accordance with a first embodiment of the present invention, in which a mixed rubidium and cesium formate brine is produced.

A mixed alum salts feed material 1 is passed to a precipitation step 20 in which contained cesium, rubidium and potassium are dissolved. Limestone slurry 2 and re-cycle solution 23 are added to this stage in which aluminium hydroxide precipitates. A precipitation discharge 4 is passed from a precipitation step 20 to a solid liquid separation step, for example a filter 21, producing a solid residue 5 and a pregnant leach solution (PLS) 6 containing the bulk of extracted cesium, rubidium and potassium.

A PLS 6 from the filter 21 is passed to the first of four extraction stages of a solvent extraction step (E1 to E4, being 24, 25, 26 and 27, respectively) in which it is contacted with stripped organic 22 in a counter-current operation. Potassium hydroxide solution 7 is injected into each stage to control the pH. The cesium and rubidium in the PLS 6 are loaded onto a phenol based extractant producing a raffinate 15, relatively free of cesium and rubidium, which exits the fourth extraction stage 27. The loaded organic 16 subsequently exits the first extraction stage 24 and is scrubbed of the loaded impurities and possibly some cesium and rubidium in two scrubbing stages 32 and 33. A scrub solution 17 enters the second scrub stage 33 and exits the first scrub stage 32 as a scrub raffinate 18. The scrub raffinate 18 is then returned to the first extraction stage 24 to recover cesium and rubidium that was scrubbed from the organic in scrub stages 32 and 33.

A scrubbed organic 19 is passed from the second scrub stage 33 to the first of three stripping stages 34, 35 and 36 of the solvent extraction circuit, in which the scrubbed organic 19 is contacted in a counter-current operation with formic acid strip liquor 20. The stripped organic 22 exits the third strip stage 36 and is recycled to the fourth extraction stage 27 to recover more cesium and rubidium. The strip liquor, being a rubidium and cesium containing brine 21, exits the first strip stage.

The raffinate 15, which contains potassium sulfate is passed to a crystalliser 37, which forces the crystallisation of potassium sulfate 24. The liquor exiting the crystalliser, recycle solution 23, is directed to the precipitation stage 20, to recover metals in this solution.

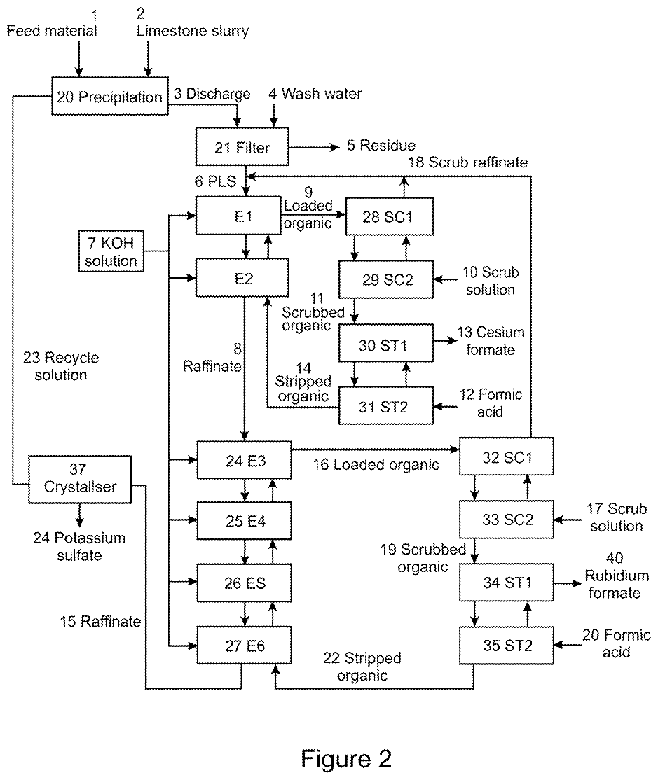

In FIG. 2 there is shown a metal recovery process in accordance with a second embodiment of the present invention in which separate cesium and rubidium formate products are produced. In as much as the process shares certain process steps shown in FIG. 1 like numerals denote like parts/steps/stages.

The PLS 6 from the filter 21 is passed to a first of two cesium extraction stages E1 and E2 of a solvent extraction step in which it is contacted with stripped organic 14 in a counter current operation. Potassium hydroxide solution 7 is injected into each stage to control the pH. The cesium in the PLS 6 is loaded onto a phenol based extractant producing a raffinate 8, relatively free of cesium, which exits the second extraction stage E2.

A loaded organic 9 subsequently exits the first extraction stage E1 and is scrubbed of the loaded impurities and possibly some cesium in two scrubbing stages 28 and 29. A scrub solution 10 enters the second scrub stage 29 and exits the first scrub stage 28. The scrub raffinate 18 is then returned to the first extraction stage E1 to recover cesium that was scrubbed from the organic.

The scrubbed organic 11 is passed from the second scrub stage 29 to the first of two stripping stages 30 and 31 of the solvent extraction circuit, in which it is contacted in a counter-current operation with formic acid strip liquor 12. A stripped organic 14 exits the second strip stage 31 and is recycled to the second extraction stage E2 to recover more cesium. A strip liquor, being a cesium formate containing brine 13, exits the first strip stage 30.

The raffinate 8 from cesium solvent extraction circuit enters the first extraction stage of the rubidium solvent extraction circuit 24. The circuitry from this point forward is consistent with the flowsheet of FIG. 1. The exception being product stream 21 contains mainly rubidium formate (as opposed to being a rubidium and cesium containing brine as shown in FIG. 1 and described hereinabove).

The present invention may be described conveniently by way of reference to the following non-limiting examples.

### Example 1

A mixed cesium, rubidium and potassium alum was prepared by leaching lepidolite in sulfuric acid and selectively crystallising the mixed salt from the leach liquor. The alum contained 6.16% K, 2.04% Rb, 0.25% Cs, 5.61% Al and 13.0% S. The alum was re-pulped in water and subject to precipitation using lime at pH 12.0. The precipitation slurry was filtered and the filtrate contained 8.10 g/L K, 3.75 g/L Rb, 0.43 g/L Cs and only 1 mg/L Al.

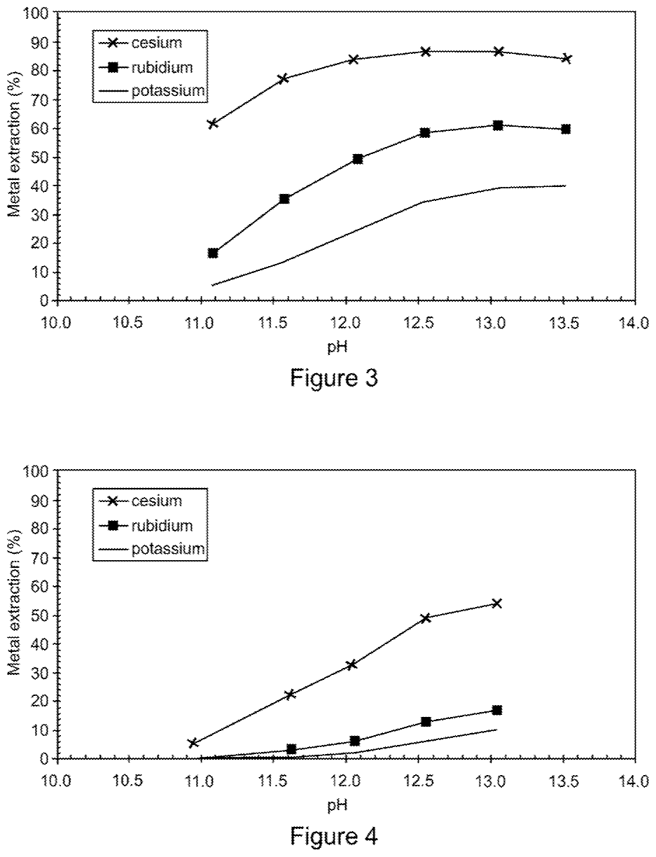

This solution was mixed with an organic solution containing 40% nonylphenol in Shellsol 2046™ at an O/A ratio of 1:1 and at different pH. Samples of the emulsion were taken at pH 11.0, 11.5, 12.0, 12.5, 13.0 and 13.5. The pH was increased using 50% KOH solution. The metal extraction vs pH is presented in FIG. 3. The data indicates excellent selectivity for cesium over rubidium and potassium and relatively good selectivity for rubidium over potassium.

### Example 2

The filtrate from Example 1 was mixed with an organic solution containing 10% nonylphenol in Shellsol 2046™ at an O/A ratio of 1:1 and at different pH. Examples of the emulsion were taken at pH 11.0, 11.5, 12.0, 12.5 and 13. The pH was increased using 50% KOH solution. The metal extraction vs pH is presented in FIG. 4. The data indicates excellent selectivity for cesium over rubidium and potassium.

### Example 3

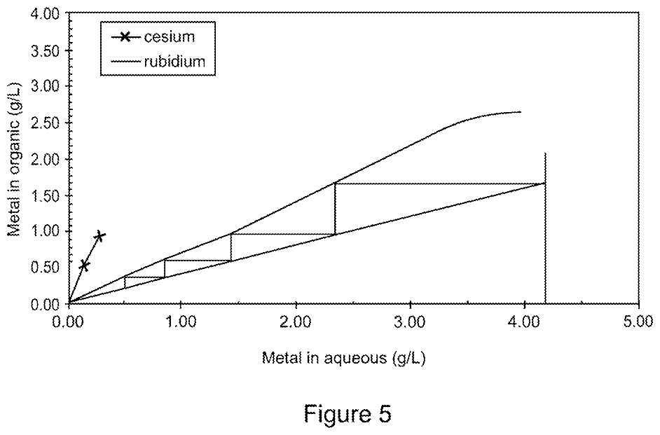

The filtrate from Example 1 was mixed with an organic solution containing 25% nonylphenol at 0/A ratios of 5:1, 3:1, 1:1, 1:3 and 1:5 at pH 12.5 for 4 minutes at room temperature. The phases were allowed to separate then filtered individually. The aqueous solutions were assayed for cesium, rubidium and potassium. The organic solutions were stripped with 10% sulfuric acid and the strip liquors were assayed for cesium, rubidium and potassium. The results are presented as a McAbe Thiele diagram in FIG. 5. The diagram indicates that >88% Rb can be extracted from the liquor in 4 stages at an advance 0/A ratio of 0.4:1, resulting in a loaded organic solution containing 0.165 g/L Cs and 1.65 g/L Rb.

As can be seen from the above description, the brine and method for producing same of the present invention, being in particular a cesium and rubidium formate brine and a method for producing same, overcome substantially the problems identified in the prior art. As noted herein, the method of the present invention is intended, in one form, for use in the recovery of cesium and rubidium, and separation of such from other monovalent cations, such as lithium, potassium and sodium, and anions such as sulfate and chloride, by solvent extraction. The process of solvent extraction described utilises phenolic functionalities, such as long chain phenols, to selectively extract cesium and rubidium from solution. Cesium and rubidium, present in the loaded organic, are recovered by stripping with formic acid to form cesium and rubidium formate brine.

Further and more particularly, the described method of the present invention relates to the recovery of cesium and rubidium for the production of a brine, the cesium and rubidium being recovered from alum salts produced from the leaching of lithium containing mica.

It is envisaged that the brines produced by the methods of the present invention will have application as a completion or drilling fluid.

Modifications and variations such as would be apparent to the skilled addressee are considered to fall within the scope of the present invention.

1.-21. (canceled)

22. A method to produce a brine from mixed alum salts, the method comprising the steps of:
(i) Dissolving or pulping alum salts containing rubidium alum, cesium alum and/or potassium alum in water or a recycled liquor and adding a neutralising agent to precipitate aluminium as aluminium hydroxide and some sulfate;
(ii) Passing the product of step (i) to a solid liquid separation stage to remove precipitated solids from step (i);
(iii) A decant or filtrate from step (ii) is passed to a solvent extraction stage whereby any contained cesium and rubidium is selectively extracted into the organic phase to form a loaded organic;
(iv) Contacting the loaded organic solution of step (iii) with a scrub solution, which is at a pH lower than the extraction pH, to effectively scrub co-loaded potassium from the organic phase;
(v) Contacting the scrubbed organic of step (iv) with formic acid to strip cesium and rubidium from the organic, the stripped cesium and rubidium forming a cesium and/or rubidium sulfate brine; and
(vi) Recycling the stripped organic of step (v) to the extraction stage.

23. The method of claim 22, wherein potassium or sodium hydroxide are added to maintain pH in the solvent extraction stage (iii) and thereby increase the extraction efficiency of rubidium and cesium.

24. The method of claim 22, wherein the active component of the organic comprises a phenolic functionality.

25. The method of claim 22, wherein the extraction order in solvent extraction stage (iii) is Cs>Rb>K>Li>Na.

26. The method of claim 22, wherein the active component of the organic is a para alkyl substituted phenol.

27. The method of claim 26, wherein the alkyl substituent:
a. contains from 9-20 carbon atoms; and
b. includes nonylphenol and dodecylphenol.

28. The method of claim 22, wherein a raffinate produced from step (iii) is contacted with organic solution and acidified liquor to recover soluble extract to the organic phase.

29. The method of claim 22, wherein a raffinate produced from step (iii) contains soluble extractant due to the high pH of the extraction stage.

30. The method of claim 29, wherein the raffinate post acidification containing a relatively high potassium rubidium and potassium/cesium ratio, is passed to a crystalliser to recover potassium sulfate.

31. The method of claim 30, wherein a solid potassium sulfate is separated from the crystallisation slurry by a solid liquid separation stage.

32. The method of claim 31, wherein a separated liquid or filtrate is recycled to step (i).

33. The method of claim 22, wherein the scrub solution from step (iv), which contains potassium and some rubidium and cesium, is recycled to the extraction stage step (iii) to recover cesium and rubidium.

34. The method of claim 22, wherein the method further comprises the separation of rubidium and potassium in an additional, initial solvent extraction stage.

35. The method of claim 22, wherein cesium is extracted from the solution prepared in step (i) in an extraction stage by which the pH and/or organic aqueous flow rates are controlled to limit any co-extraction of rubidium and potassium.

36. The method of claim 35, wherein a loaded organic containing cesium and some rubidium is passed to a separate scrubbing stage conducted at a pH lower than the extraction pH, in which co-loaded rubidium is scrubbed from the organic solution, a scrub solution containing rubidium optionally being recycled to the extraction stage, and the scrubbed organic further optionally being stripped with formic acid to produce a relatively pure cesium formate brine.

37. The method of claim 34, wherein a raffinate produced from the initial solvent extraction stage is subject to extraction in accordance with step (iii) and the subsequent scrubbing and stripping stages in accordance with steps (iv) and (v) to produce a relatively pure rubidium formate brine.

38. A brine containing one or both of cesium formate and rubidium formate produced by the method of claim 22.

39. The brine of claim 38, wherein the brine has a specific gravity of greater than about 1.7.

40. A completion or drilling fluid comprising the brine of claim 38.
